(19) United States
(12) Patent Application Publication
(10) Pub. No.: US 2025/0268531 A1
(43) Pub. Date: Aug. 28, 2025

(54) ADVANCED MEASUREMENT MODES FOR A WEARABLE DEVICE

(71) Applicant: Oura Health Oy, Oulu (FI)

(72) Inventors: Mika Petteri Kangas, Oulu (FI); Henri Matti Johannes Huttunen, Pudasjärvi (FI); Jaakko Tapio Vartiainen, Oulu (FI)

(21) Appl. No.: 18/584,800

(22) Filed: Feb. 22, 2024

(51) Int. Cl. A61B 5/00; G06F 11/34; H04W 76/14

(52) U.S. Cl. CPC A61B 5/6802; G06F 11/3438; H04W 76/14

(57) ABSTRACT

Methods, systems, and devices for operating a wearable device are described. While operating in a first measurement mode, a wearable device may determine a baseline setting for a measurement parameter for collecting biometric data from a user that satisfies a first quality metric and a first power consumption metric. As part of switching from the first measurement mode to a second measurement mode, the wearable device may update the measurement parameter from the baseline setting to a second setting. The second setting may be based on a second power consumption metric associated with the second measurement mode and may be based on biometric data collected for the user satisfying a second quality metric associated with the second measurement mode.

### FIELD OF TECHNOLOGY

The following relates to wearable devices and data processing, including advanced measurement modes for a wearable device.

### BACKGROUND

Some wearable devices may be configured to collect biometric data from users using various sensors. Accordingly, the wearable devices may support one or more measurement modes that control the settings of various measurement parameters. Improved techniques for wearable devices with measurement modes may be desired.

### BRIEF DESCRIPTION OF THE DRAWINGS

FIG. 1 illustrates an example of a system that supports advanced measurement modes for a wearable device in accordance with aspects of the present disclosure.

FIG. 2 illustrates an example of a system that supports advanced measurement modes for a wearable device in accordance with aspects of the present disclosure.

FIG. 3 shows an example of a system that supports advanced measurement modes for a wearable device in accordance with aspects of the present disclosure.

FIG. 4 shows an example of a wearable device that supports advanced measurement modes in accordance with aspects of the present disclosure.

FIG. 5 shows an example of a process flow that supports advanced measurement modes for a wearable device in accordance with aspects of the present disclosure.

FIG. 6 shows a block diagram of an apparatus that supports advanced measurement modes in accordance with aspects of the present disclosure.

FIG. 7 shows a block diagram of a wearable device manager that supports advanced measurement modes for a wearable device in accordance with aspects of the present disclosure.

FIG. 8 shows a diagram of a system including a device that supports advanced measurement modes in accordance with aspects of the present disclosure.

FIG. 9 shows a flowchart illustrating methods that support advanced measurement modes for a wearable device in accordance with aspects of the present disclosure.

### DETAILED DESCRIPTION

A wearable device configured to collect biometric data from a user may have one or more static, device-selectable measurement modes that control the settings of various measurement parameters. For example, each static measurement mode may specify a fixed combination of settings for the measurement parameters. But the performance of the wearable device using the device-selectable, static measurement modes may not be specific to the interests or the physiological individuality of the user. According to the techniques described herein, a wearable device may support user-selectable, adaptive measurement modes that allow the user to control the performance of the wearable device and that account for user-specific physiological characteristics (e.g., skin color, resting heart rate, nocturnal restlessness).

Aspects of the disclosure are initially described in the context of systems supporting physiological data collection from users via wearable devices. Additional aspects of the disclosure are described with reference to a wearable device and process flows. Aspects of the disclosure are further illustrated by and described with reference to apparatus diagrams, system diagrams, and flowcharts that relate to advanced measurement modes for a wearable device.

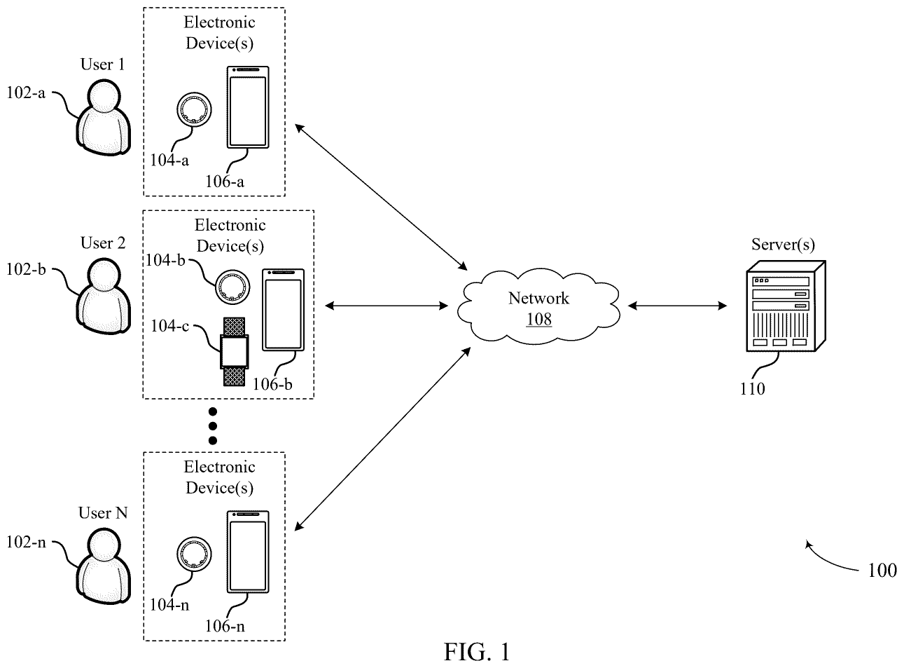

FIG. 1 illustrates an example of a system 100 that supports advanced measurement modes for a wearable device in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

So, a wearable device 104 may be configured to collect biometric data from a user. In addition to one or more static, device-selectable measurement modes, the wearable device 104 may support one or more advanced (e.g., user-selectable, adaptive) measurement modes that control the settings of various measurement parameters in a user-specific manner. The advanced measurement modes may be user-selectable and may be associated with different performance metrics, such as battery consumption and the type and quality of biometric data collected, so that the user 104 can tailor the performance of the wearable device to the interests of the user 104. The advanced measurement modes may be adaptive in that the measurement modes may dynamically select the settings of measurement parameters based on biometric data collected for the user (rather than having fixed settings per measurement mode) and the performance metrics associated with the respective measurement modes.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

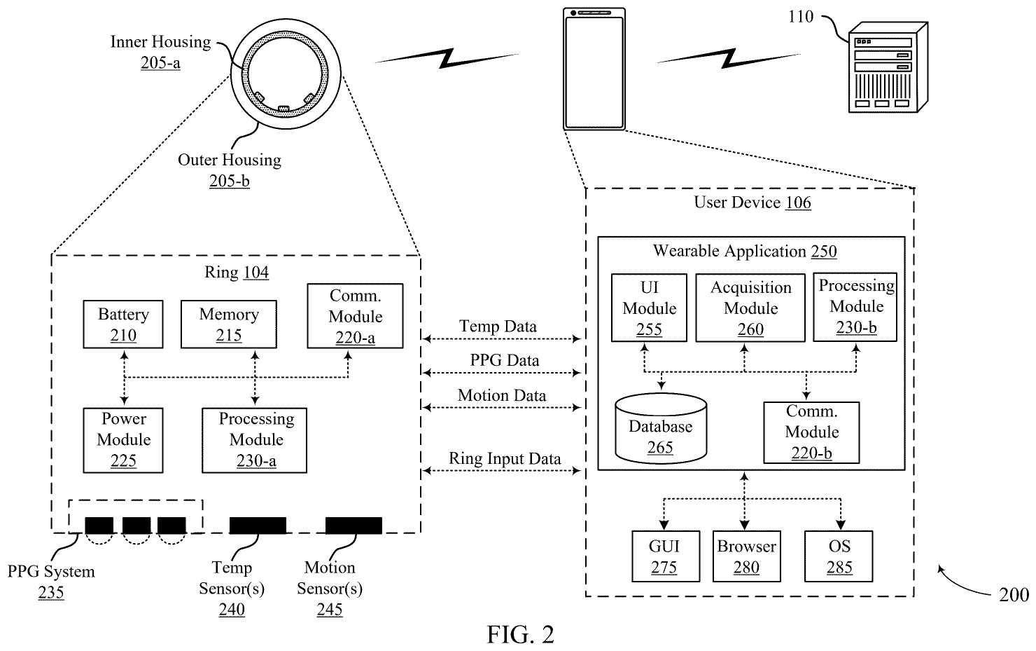

FIG. 2 illustrates an example of a system 200 that supports advanced measurement modes for a wearable device in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

The ring 104 (or other wearable device) may support different operating modes that are associated with different baseline performance metrics and different baseline measurement modes. For example, the ring 104 (or other wearable device) may support, among additional or different operating modes, a sleep operating mode that prioritizes the collection of sleep-related biometric data, a workout operating mode that prioritizes the collection of activity-related biometric data, and an SpO2 operating mode that prioritizes the collection of blood oxygen-related biometric data. Accordingly, the ring 104 (or other wearable device) may use different baseline measurement modes for the different operating modes.

The baseline measurement mode for an operating mode may be associated with baseline performance metrics (e.g., battery consumption, type of biometric data collected, quality of biometric data collected) and thus may have associated baseline measurement parameter settings that achieve the performance metrics. The baseline measurement parameters settings may be user-agnostic (e.g., predefined, fixed, static) or may be user-specific (e.g., dynamically determined based on biometric data collected for the user).

In addition to supporting the baseline measurement mode, an operating mode may support one or more advanced measurement modes that are associated with different performance metrics (and thus different measurement parameter settings). The advanced measurement modes may be user-specific in that the measurement parameter settings may be dynamically determined based on biometric data collected for the user. In some examples, the performance metric(s) for an advanced measurement mode may also be user-specific. For example, the performance metric(s) may be based on user interaction with an application of the user device 106 or may be directly selected by the user. The ring 104 (or other wearable device) may switch from the baseline measurement mode for an operating mode to an advanced measurement mode for the operating mode autonomously (e.g., without a prompt from the user) or in response to a prompt from the user.

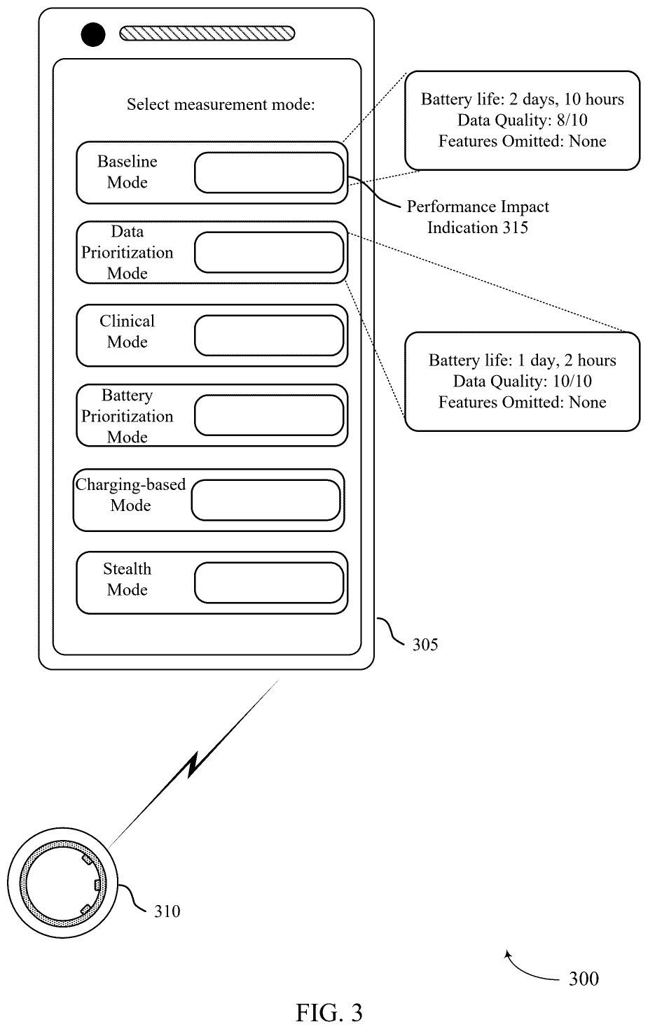

FIG. 3 shows an example of a system 300 that supports advanced measurement modes for a wearable device in accordance with aspects of the present disclosure. The system 300 may include a wearable device 310 that supports various measurement modes, including a baseline mode and one or more advanced measurement modes, which may vary across operating modes. The wearable device 310 may be in wireless communication with the user device 305 and the two devices may exchange information related to the measurement modes of the wearable device 310. The user device 305 may also display (e.g., via a graphical user interface) information related to the various measurement modes supported by the wearable device 310. For example, the user device 305 may display (concurrently or at different times) one or more measurement modes for selection by the user. The user device 305 may also display indications 315 of the performance impacts of the measurement modes on the wearable device 310.

The wearable device 310 may support a baseline measurement mode that is associated with operating mode-specific performance metrics and operating mode-specific measurement parameter settings. Regardless of the operating mode, the measurement parameter settings of the baseline measurement mode may balance battery consumption with data quality and may provide a default set of features (e.g., types of data collected). For example, for a given operating mode, the measurement parameter settings of the baseline measurement mode may provide a baseline quality of data at (or under) a baseline power consumption level. In some examples, the baseline measurement mode may be the default measurement mode of the wearable device 310. A measurement parameter setting may also be referred to as a measurement setting or other suitable terminology.

The user device 305 may display an indication 315 of the performance impact of the baseline measurement mode. The performance impact may include a power consumption metric (e.g., remaining battery life), a data quality metric (e.g., on a scale of 1-10, with 1 being the minimum quality supported by the wearable device 310 and 10 being the maximum quality supported by the wearable device 310), and/or a list of included feature (e.g., type of biometric data collected) or omitted features (e.g., types of biometric data not collected, processing not performed, types of user insights not provided) associated with the baseline measurement mode. The performance impact may be based on performance information received from the wearable device 310.

The wearable device 310 may also support one or more advanced measurement modes. The advanced measurement modes may device-selectable or user-selectable and the measurement parameter settings of the advance measurement modes may be user-specific and/or operating mode-specific.

In some examples, the advanced measurement modes may include a Data Prioritization mode that prioritizes data quality (e.g., of all supported biometric data types) at the expense of battery life. For example, for a given operating mode, the Data Prioritization mode may be associated with 1) higher data quality metrics than the baseline measurement mode and 2) a higher power consumption metric than the baseline measurement mode. A first data quality metric may be referred to as “higher” than a second data quality metric if the first data quality metric is associated with better quality data (e.g., data with higher signal-to-noise ratio (SNR)). A first power consumption metric may be referred to as a “higher” than a second power consumption metric if the first power consumption metric is associated with more power consumption (e.g., more current use). A power consumption metric for a measurement mode may represent the maximum amount of power the wearable device 310 is permitted to consume in that measurement mode.

In some examples, the advanced measurement modes may include a Clinical Mode that prioritizes the data quality of a subset biometric data types (but not others) at the expense of battery life. For instance, the Clinical Mode may prioritize the quality of Sp02 data over the quality of other biometric types. Thus, for a given operating mode, the Clinical Mode may be associated with 1) higher data quality metric(s) than the baseline measurement mode for a first subset of biometric data types, 2) equal or lower data quality metric(s) than the baseline measurement mode for a second subset of biometric data types, and 3) a higher power consumption metric than the baseline measurement mode. In some examples, the data quality metric for a type of biometric data included in the first subset of biometric data types may be the highest quality setting supported by the wearable device and may provide diagnostic-grade quality. The types of biometric data prioritized in the Clinical Mode may be selectable by the user or may be based on indicia of a user interest level in the types of biometric data. In some examples, the Clinical Mode may be a sub-mode of the Data Prioritization Mode. In some examples, the Clinical Mode may feature a more advanced data processing pipeline than other measurement modes. For example, the wearable device 310 may collect raw PPG data from multiple different signal channels and may transfer the raw PPG data to another device (e.g., the user device 305, a server) for more advanced processing. In such examples, the Clinical Mode may be associated with higher memory consumption at the wearable device 310 and higher communication-related power consumption.

In some examples, the advanced measurement modes may include a Battery Prioritization mode that prioritizes battery life at the expense of data quality. For example, for a given operating mode, the Battery Prioritization mode may be associated with 1) lower data quality metric(s) than the baseline measurement mode and 2) a lower power consumption metric than the baseline measurement mode. A first data quality metric may be referred to as “lower” than a second data quality metric if the first data quality metric is associated with worse quality data (e.g., data with a lower SNR). A first power consumption metric may be referred to as a “lower” than a second power consumption metric if the first power consumption metric is associated with less power consumption (e.g., less current use).

In some examples, the advanced measurement modes may include a Charging-based Mode that balances data quality and battery life based on an anticipated (e.g., expected, predicted) time for charging the wearable device 310. The wearable device 310 may dynamically select performance metrics (and measurement parameters settings that achieve the selected performance metrics) for the Charging-based Mode based on user feature-interest, a remaining battery life of the wearable device 310, the anticipated time for charging the wearable device 310, or any combination thereof. User feature-interest may refer to an interest level of the user in one or more features (e.g., types of biometric data). As an example, the wearable device 310 may select a first subset of biometric data types to prioritize over a second subset of biometric data types based on the user's relative interest levels in the biometric data types. The wearable device 310 may determine the anticipated time for charging the wearable device 310 based on an indication of the time from the user, a past charging pattern for the wearable device 310 (e.g., as detected by the wearable device 310), or both.

In some examples, the advanced measurement modes may include a Stealth Mode that prioritizes the reduction of light pollution over data quality. For example, the Stealth Mode may be associated with measurement parameter settings that reduce the amount of visible light emitted by the wearable device 310. As an illustration, in the Stealth Mode the wearable device 310 may configure the optical transmitters of the wearable device 310 to output infrared signals instead of visible light signals. Because the Stealth Mode limits the functionality of the optical transmitters, the Stealth Mode may have 1) lower data quality metric(s) than the baseline measurement mode, and/or 2) a lower power consumption metric than the baseline measurement mode.

The measurement parameter settings of an advanced measurement mode (which may be used to achieve the performance metrics of that advanced measurement mode) may be user-specific (as opposed to user-agnostic), which may tailor the performance of the wearable device 310 to the user. For example, the setting for a given measurement parameter may be dynamically determined by the wearable device 310 based on biometric data collected for the user. The biometric data may be historic biometric data (e.g., biometric data collected before the wearable device 310 enters the advance measurement mode), calibration biometric data (e.g., biometric data collected in the advanced measurement mode), or both.

As an illustration, upon entering an advanced measurement mode, the wearable device 310 may iteratively change the measurement parameters settings and evaluate biometric data collected using the different measurement parameter settings to determine the combination of measurement parameter settings that achieves the performance metrics for the advanced measurement mode. As another illustration, the wearable device 310 may use historic biometric data to determine the performance metrics associated with different combinations of measurement parameter settings. For instance, the wearable device 310 may use historic biometric data that indicates the user has a low resting heartrate at night to reduce (e.g., while the user is asleep) the sample rate (e.g., relative to the baseline sample rate) of the sensors used to collect heartrate information.

In some examples, the performance metrics of an advanced measurement mode may be user-specific. For example, the quality metric (e.g., for a type of biometric data) of an advanced measurement setting may be selected based on an interest level of the user for that type of biometric data. The wearable device 310 may gauge the user's interest level in a type of biometric data based on an interaction pattern of the user with the application (e.g., how often the user checks the information related to the type of biometric data) or based on an indication of the user's interest level received (e.g., by the user device 305) as a user input from the user. As another example, the power consumption metric of the advanced measurement setting may be selected based on power consumption profile of the user, based on an anticipated next charging time for the wearable device 310, based on a past charging pattern for the wearable device 310, or any combination thereof.

Thus, the system 300 may support advanced measurement modes that have user-specific measurement parameter settings that tailor measurements to the user.

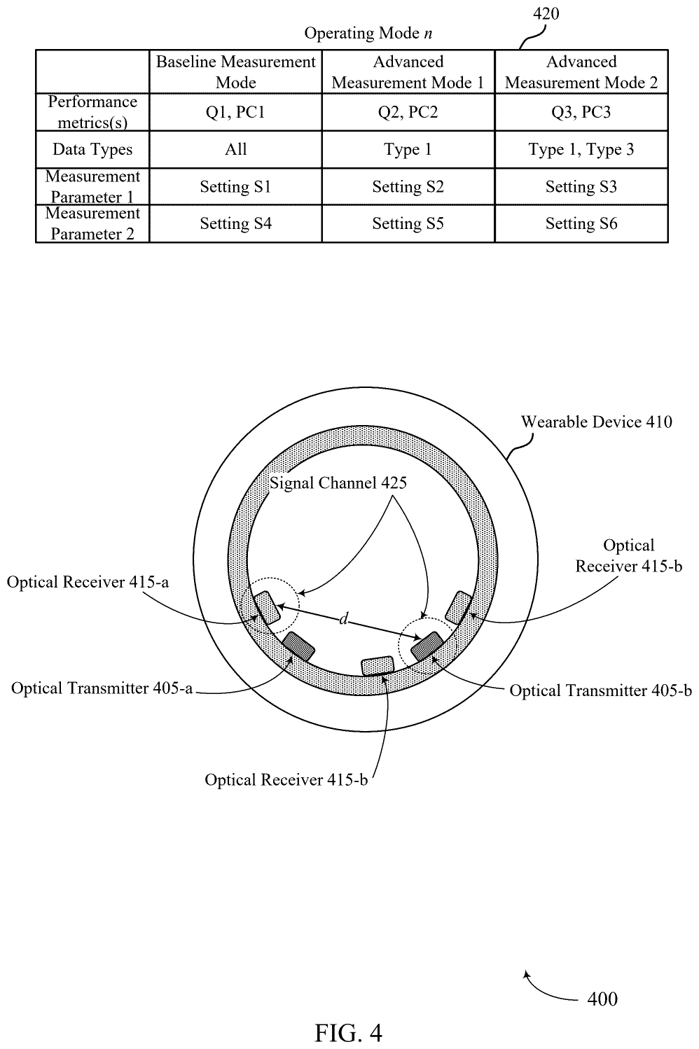

FIG. 4 shows an example of a wearable device 410 that supports advanced measurement modes in accordance with aspects of the present disclosure. The wearable device 410 may support one or more operating modes, including operating mode n, as well as various measurement modes for the operating modes, including a baseline measurement mode and multiple advanced measurement modes (e.g., Advanced Measurement Mode 1, Advanced Measurement mode 2).

Table 420 shows, for operating mode n, the different performance metrics, types of biometric data collected, and measurement parameter settings for the baseline mode, Advanced Measurement Mode 1, and Advanced Measurement mode 2. Across different operating modes the same measurement mode may have different associated performance metrics and measurement parameter settings. So, switching between operating modes (while staying in the same measurement mode) may involve adjusting (e.g., modifying) one or more measurement parameter settings to achieve the performance metrics for the new operating mode.

As illustrated, the baseline measurement mode for operating mode n may be associated with a quality metric Q1 (e.g., in units of SNR) and a power consumption metric PC1 (e.g., in units of current) and may collect all types of biometric data (e.g., N types) supported by the wearable device 410. Although described with reference to a single quality metric Q1, which may be a shared or average data quality metric across the types of biometric data, the baseline measurement mode may have different data quality metrics for different types of biometric data (e.g., there may be a respective data quality metric for each type of biometric data collected in the baseline measurement mode). To achieve the performance metrics (e.g., Q1 and PC1), the baseline measurement mode may have associated measurement parameter settings (e.g., setting S1, setting S4). The measurement parameter settings of the baseline measurement mode may be user-agnostic or user-specific (e.g., based on biometric data collected for the user). Examples of measurement parameters and settings are described below.

Advanced Measurement Mode 1 for operating mode n may be associated with a quality metric Q2 (e.g., in units of SNR) and a power consumption metric PC2 (e.g., in units of current) and may collect a subset (e.g., type 1) of the types of biometric data supported by the wearable device 410. The performance metrics associated with Advanced Measurement Mode 1 may be based on the performance metrics for the baseline mode (e.g., Q2 may be x % of Q1, PC2 may be y % of PC1). Although described with reference to a single quality metric Q2, which may be a shared or average data quality metric across the types of biometric data, Advanced Measurement Mode 1 may have different data quality metrics for different types of biometric data (e.g., there may be a respective data quality metric for each type of biometric data collected in Advanced Measurement Mode 1). To achieve the performance metrics (e.g., Q2 and PC2), Advanced Measurement Mode 1 may have associated measurement parameter settings (e.g., setting S2, setting S5). The measurement parameter settings of Advanced Measurement Mode 2 may be user-specific (e.g., based on biometric data collected for the user).

Advanced Measurement Mode 2 for operating mode n may be associated with a quality metric Q3 (e.g., in units of SNR) and a power consumption metric PC3 (e.g., in units of current) and may collect a subset (e.g., type 1, type 3) of the types of biometric data supported by the wearable device 410. The performance metrics associated with Advanced Measurement Mode 2 may be based on the performance metrics for the baseline mode. Although described with reference to a single quality metric Q3, which may be a shared or average data quality metric across the types of biometric data, Advanced Measurement Mode 1 may have different data quality metrics for different types of biometric data (e.g., there may be a respective data quality metric for each type of biometric data collected in Advanced Measurement Mode 2). To achieve the performance metrics (e.g., Q3 and PC3), Advanced Measurement Mode 2 may have associated measurement parameter settings (e.g., setting S3, setting S6). The measurement parameter settings of Advanced Measurement Mode 2 may be user-specific (e.g., based on biometric data collected for the user).

In some examples, the performance metrics associated with a measurement mode may be based on charging information for the wearable device 410. For example, the wearable device 410 may select data quality metric(s), a power consumption metric, or both, based on an anticipated time for the wearable device 410 to be charged (referred to as the anticipated charging time). Additionally or alternatively, the type(s) of biometric data collected in a measurement mode may be based on the anticipated charging time. In some examples, the performance metrics associated with a measurement mode may be based on the interests of the user.

The wearable device 410 may achieve the performance metrics for a given measurement mode by adjusting (relative to a different measurement mode) the settings of one or more measurement parameters. The measurement parameters may be software parameters, hardware parameters (e.g., sensor-related parameters), or both.

As an example, the wearable device 410 may adjust the amount of current supplied to one or more sensors (e.g., one or more optical components. For instance, the wearable device 410 may adjust the amount of current supplied to one or more optical transmitters 405, which in turn may adjust the intensity of the light signals output by the one or more optical transmitters 405. The intensity of the light signals may be proportionally related to 1) the quality of the data that is collected based on the light signals and 2) the power consumption of the wearable device 410. For instance, increasing the intensity of a light signal may increase the quality of the data as well as increase the power consumption of the wearable device 410, whereas decreasing the intensity may decrease the quality of the data and decrease the power consumption of the wearable device 410.

As another example, the wearable device 410 may achieve one or more target performance metric(s) by adjusting the activation status of one or more optical transmitters 405, one or more optical receivers 415, or both. The activation status of an optical component (e.g., an optical transmitter 405, an optical receiver 415) may refer to whether the optical component is activated (e.g., enabled) or deactivated (e.g., disabled). Activating an optical component may increase the quality of data as well as increase the power consumption of the wearable device 410, whereas deactivating an optical component may decrease the quality of the data and decrease the power consumption of the wearable device 410.

As another example, the wearable device 410 may achieve one or more target performance metrics by adjusting the activation status of one or more signal channels, where a signal channel includes a combination of optical components through which a signal (e.g., an optical signal) propagates. For example, signal channel 425 may include optical transmitter 405-b, which may output an optical signal, and optical receiver 415-a, which may receive the optical signal (e.g., after reflection of the optical signal through the user's finger). Use of longer signal channels may increase data quality at the cost of increasing power consumption, where the length of a signal channel refers to the distance d between the components of the signal channel.

So, the wearable device 410 may increase data quality (and increase power consumption) by activating longer signal channels and may decrease data quality (and decrease power consumption) by deactivating longer signal channels. Thus, the length of a signal channel may be used as a basis for activating/deactivating that signal channel, where activating a signal channel refers to activating the combination of optical components that make up that signal channel and where deactivating the signal channel refers to deactivating the combination of optical components that make up that signal channel.

As another example, the wearable device 410 may achieve one or more target performance metrics by adjusting the type(s) of optical signal output by one or more optical transmitters 405. For instance, the wearable device 410 may configure an optical transmitter 405 to output a color of light (e.g., green light) that is associated with a different data quality, different power consumption, or both, relative to a different color of light (e.g., red light). As another illustration, the wearable device 410 may configure an optical transmitter 405 to output infrared signals instead of visible light signals.

As another example, the wearable device 410 may achieve one or more target performance metrics by adjusting the sample rate of a sensor (e.g., an optical receiver 415). For example, the wearable device 410 may decrease power consumption, at the expense of data quality, by decreasing the rate at which an optical receiver 415 samples (e.g., measures) received optical signals. Additionally or alternatively, the wearable device 410 may adjust the sample rate of a sample circuit that is configured to sample (e.g., measure) the signals received by the optical receivers 415.

As another example, the wearable device 410 may achieve one or more target performance metrics by adjusting scanning operations for one or more signal channels. For example, the wearable device may adjust the scan rate(s) (e.g., frequency) of one or more signal channel(s), where the scan rate of signal channel refers to the rate at which the wearable device 410 performs scanning operations for a signal channel. A scanning operation for a signal channel may include activation and performance evaluation of the signal channel with different settings (e.g., types of optical signals, intensity of optical signals, sample rates) for the optical components included in the signal channel. Decreasing the scanning rate of a signal channel may decrease the quality of data collected using that signal channel (and may decrease the power consumption of the wearable device 410), whereas increasing the scanning rate of a signal channel may increase the quality of data collected using that signal channel (and may increase the power consumption of the wearable device 410). The wearable device 410 may adjust the scan rate of the signal channels collectively (e.g., as a set) or the wearable device 410 may adjust individual scan rates for the signal channels.

Additionally or alternatively, the wearable device 410 may adjust the scan settings used for the optical components of a signal path (e.g., the wearable device 410 may omit high confidence colors of light from the scanning operations, where high confidence colors of light refer to colors of light for which data quality is consistently within a threshold range). Additionally or alternatively, the wearable device 410 may adjust the signal paths scanned by the wearable device (e.g., the wearable device 410 may omit high confidence signal paths from scanning operations, where high confidence signal channels refer to signal channels for which data quality is consistently within a threshold range).

Thus, the wearable device 410 may adjust one or more measurement parameters settings to achieve the performance metrics associated with a measurement mode such as an advanced measurement mode.

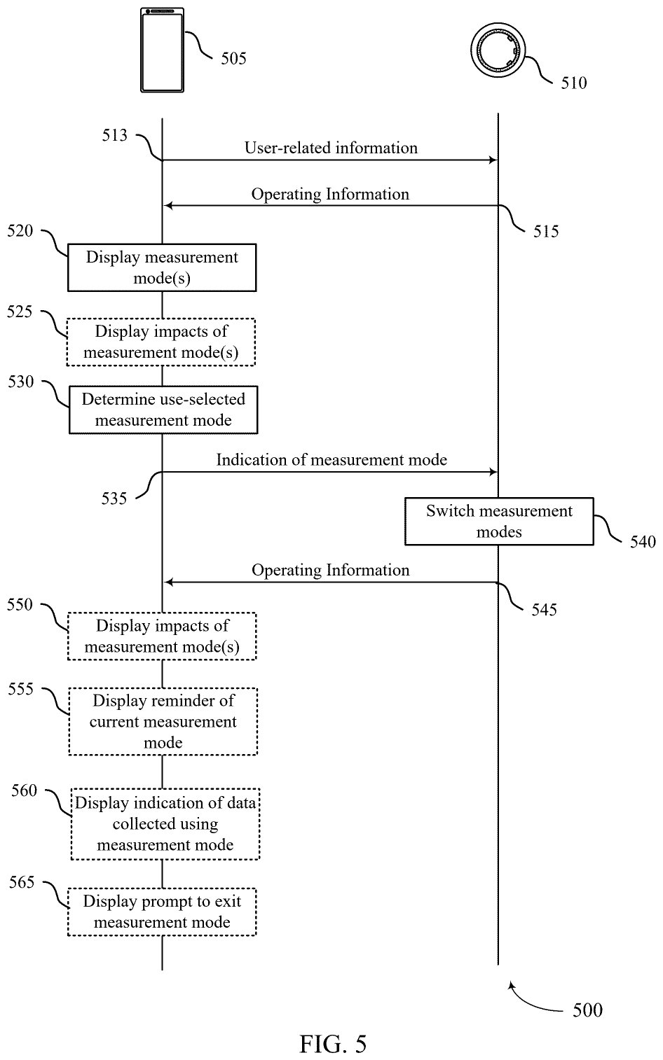

FIG. 5 shows an example of a process flow 500 that supports advanced measurement modes for a wearable device in accordance with aspects of the present disclosure. Aspects of the process flow 500 may be performed by a user device 505 and a wearable device 510. The user device 505 may have a wireless connection with the wearable device 510 such that wireless communications (e.g., over-the-air, electromagnetic signals) can be exchanged between the user device 505 and the wearable device 510. The wearable device 510 may support one or more advanced measurement modes as described herein.

Before 513, the wearable device 510 may be in an operating mode and may be using a baseline measurement mode. The baseline measurement mode may have associated baseline performance metrics, such as a baseline data quality metric and a baseline power consumption metric, and associated baseline measurement parameters settings for achieving the baseline performance metrics. The baseline measurement parameter settings may be user-agnostic (e.g., default metrics) or user-specific (e.g., based on biometric data collected from the user). Although described with reference to the baseline measurement mode as the initial measurement mode, the process flow 500 may be used to switch between any two measurement modes, including from an advanced measurement mode to another advanced measurement mode, and from an advanced measurement mode to the baseline measurement mode.

At 513, the user device 505 may transmit user-related information to the wearable device 510. In some examples, the user-related information may indicate an interest level of the user in one or more types of biometric data. In some examples, the user-related information may indicate an anticipated charging time for the user device (e.g., as indicated by the user). For example, the user may indicate a duration until the next time the wearable device 510 is charged. The wearable device 510 may use the user-related information as a basis for selecting performance metrics for one or more advanced measurement modes.

At 515, the wearable device 510 may transmit to the user device 505 operating information for the wearable device 510 as determined by the wearable device 510. The operating information may include an indication of one or more performance metrics of the wearable device 510. For example, the operating information may indicate a measurement mode of the wearable device 510 (e.g., a baseline measurement mode), a data quality associated with the measurement mode, a remaining battery life of the wearable device 510 in the measurement mode, or any combination thereof. Additionally or alternatively, the operating information may indicate one or more advanced measurement modes of the wearable device 510, data quality associated with the advanced measurement modes, a remaining battery life of the wearable device 510 in the advanced measurement modes, or any combination thereof. In some examples, the operating information may indicate charging information for the wearable device 510 (e.g., a past charging pattern for the wearable device 510, an anticipated time for charging the wearable device 510). In some examples, the operating information may indicate a recommended advanced measurement mode for the wearable device 510.

At 520, the user device 505 may display (e.g., via a graphical user interface) an indication of one or more advanced measurement modes supported by the wearable device 510. The indication may prompt the user to select an advanced measurement mode. The information displayed at 520 may be based on the operating information received at 515.

At 525, the user device 505 may display (e.g., via a graphical user interface) performance metrics associated with the current (e.g., baseline) measurement mode, performance metrics (e.g., data quality, remaining battery life, types of biometric data collected, types of biometric data not collected, processing performed, processing not performed, types of user insights provided, types of user insights not provided) for one or more selectable advanced measurement modes, or both. In some examples, the user device 505 may display a comparison of performance metrics for the current mode and one or more advanced measurement modes to aid selection by the user. Thus, the user device 505 may display the performance impact of the one or more advanced measurement modes. The information displayed at 525 may be based on the operating information received at 515.

At 530, the user device 505 may determine (e.g., based on a user input) an advanced measurement mode selected by the user. Alternatively, the user device 505 may select the advance measurement mode autonomously (e.g., without input from the user). In another alternative, the wearable device 510 may autonomously select the advanced measurement mode and indicate the selection to the user device 505. In examples where the advanced measurement mode is not selected by the user, the indication of the selected advanced measurement mode and associated performance impact may be displayed after 530 (e.g., to alert the user of the changes). At 535, the user device 505 may transmit an indication of the selected advanced measurement mode to the wearable device 510.

At 540, the wearable device 510 may switch from the baseline measurement mode to the advanced measurement mode. Switching to the advanced measurement mode may include adjusting one or more measurement parameter settings to achieve one or more performance metrics associated with the advanced measurement mode. The measurement parameter settings may be user-specific in that the measurement parameter settings are selected based on the measurement parameter settings satisfying the performance metrics associated with the advanced measurement mode. For example, the wearable device 510 may select the measurement parameter settings for the advanced measurement mode based on 1) biometric data collected using the measurement parameter settings satisfying the data quality metric(s) associated with the advance measurement mode and/or 2) the wearable device 510 satisfying the power consumption metric during collection of the biometric data using the measurement parameter settings.

At 545, the wearable device 510 may transmit to the user device 505 (e.g., based on switching to the advanced measurement mode) operating information related to the advanced measurement mode as determined by the wearable device 510. The operating information may include an indication of one or more performance metrics of the wearable device 510 using the advanced measurement mode. For example, the operating information may indicate a data quality associated with the advanced measurement mode, a remaining battery life of the wearable device 510 in the advanced measurement mode, or any combination thereof. The operating information may be updated relative to the operating information transmitted at 515. In some examples, the operating information may indicate a recommended advanced measurement mode for the wearable device 510.

At 550, the user device 505 may display (e.g., via a graphical user interface) an indication of the performance metrics received at 545. For instance, the user device 505 may display the performance impact of the one or more advanced measurement modes. The information displayed at 525 may be based on the operating information received at 545.

At 555, the user device 505 may display (e.g., via a graphical user interface) a reminder that the current measurement mode in use by the wearable device is the advance measurement mode. At 560, the user device 505 may display (e.g., via a graphical user interface) an indication of data collected using the advanced measurement mode. For example, the user device 505 may track the biometric data collected using the advance measurement mode and flag that biometric data so that the user knows the measurement conditions used to collect the biometric data.

At 560, the user device 505 may display a prompt for the user to select a different measurement mode (e.g., the baseline mode, another advance measurement mode) than the advanced measurement mode. The prompt may be based on the operating information received at 545. If the user device 505 determines that the user has selected a different measurement mode, the user device 505 may transmit an indication of the different measurement mode to the wearable device 510. Alternatively, the user device 505 may autonomously select (e.g., based on the operating information received at 515) the different measurement mode for the wearable device 510.

Thus, the user device 505 and the wearable device 510 may work together to facilitate the wearable device 510 switching between measurement modes. Alternative examples of the foregoing may be implemented, where some operations are performed in a different order than described, are performed in parallel, or are not performed at all. In some cases, operations may include additional features not mentioned herein, or further operations may be added. Additionally, certain operations may be performed multiple times or certain combinations of operations may repeat or cycle.

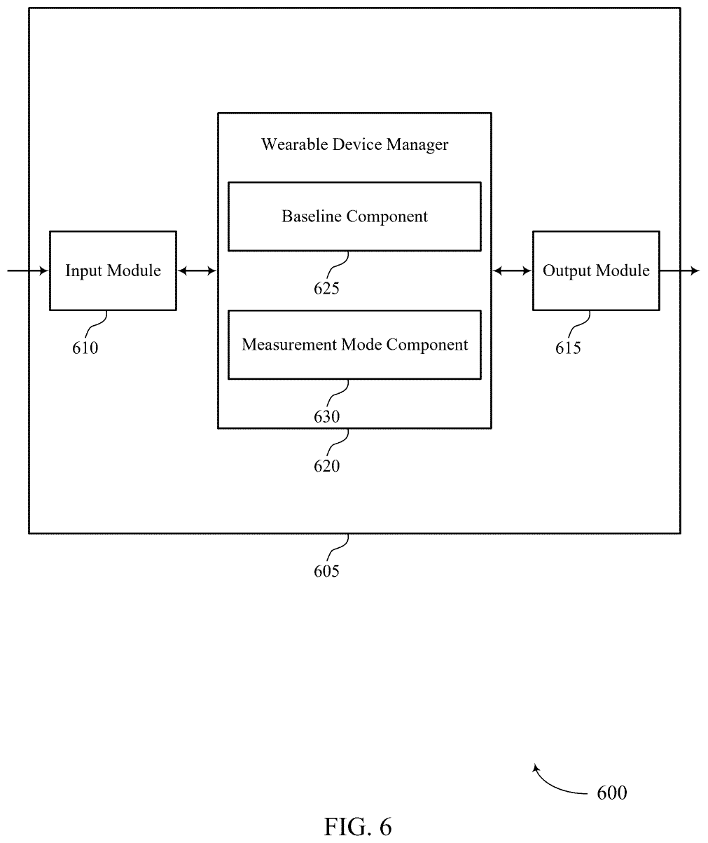

FIG. 6 shows a block diagram 600 of a device 605 that supports advanced measurement modes for a wearable device in accordance with aspects of the present disclosure. The device 605 may include an input module 610, an output module 615, and a wearable device manager 620. The device 605, or one of more components of the device 605 (e.g., the input module 610, the output module 615, the wearable device manager 620), may include at least one processor, which may be coupled with at least one memory, to support the described techniques. Each of these components may be in communication with one another (e.g., via one or more buses).

For example, the wearable device manager 620 may include a baseline component 625 a measurement mode component 630, or any combination thereof. In some examples, the wearable device manager 620, or various components thereof, may be configured to perform various operations (e.g., receiving, monitoring, transmitting) using or otherwise in cooperation with the input module 610, the output module 615, or both. For example, the wearable device manager 620 may receive information from the input module 610, send information to the output module 615, or be integrated in combination with the input module 610, the output module 615, or both to receive information, transmit information, or perform various other operations as described herein.

The baseline component 625 may be configured as or otherwise support a means for determining, for a user while the wearable device is operating in a first measurement mode, a baseline setting for a measurement parameter for collecting biometric data from the user that satisfies a first quality metric and a first power consumption metric. The measurement mode component 630 may be configured as or otherwise support a means for updating, as part of switching from the first measurement mode to a second measurement mode, the measurement parameter from the baseline setting to a second setting that is based at least in part on a second quality metric associated with the second measurement mode, a second power consumption metric associated with the second measurement mode, or both, and wherein the second setting is further based at least in part on first biometric data collected for the user satisfying the second quality metric.

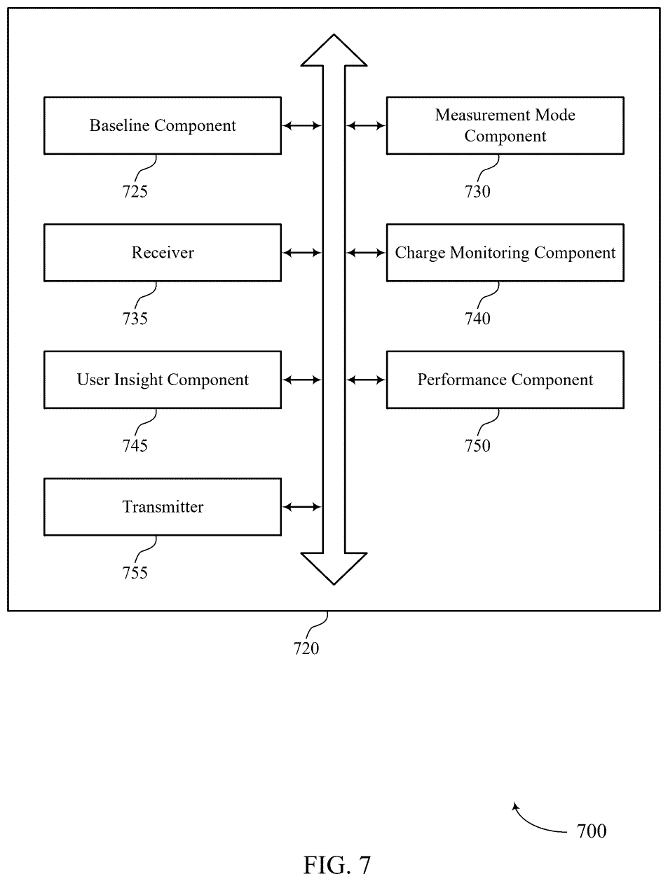

FIG. 7 shows a block diagram 700 of a wearable device manager 720 that supports advanced measurement modes for a wearable device in accordance with aspects of the present disclosure. The wearable device manager 720 may be an example of aspects of a wearable device manager or a wearable device manager 620, or both, as described herein. The wearable device manager 720, or various components thereof, may be an example of means for performing various aspects of advanced measurement modes for a wearable device as described herein. For example, the wearable device manager 720 may include a baseline component 725, a measurement mode component 730, a receiver 735, a charge monitoring component 740, a user insight component 745, a performance component 750, a transmitter 755, or any combination thereof. Each of these components, or components of subcomponents thereof (e.g., one or more processors, one or more memories), may communicate, directly or indirectly, with one another (e.g., via one or more buses).

The baseline component 725 may be configured as or otherwise support a means for determining, for a user while the wearable device is operating in a first measurement mode, a baseline setting for a measurement parameter for collecting biometric data from the user that satisfies a first quality metric and a first power consumption metric. The measurement mode component 730 may be configured as or otherwise support a means for updating, as part of switching from the first measurement mode to a second measurement mode, the measurement parameter from the baseline setting to a second setting that is based at least in part on a second quality metric associated with the second measurement mode, a second power consumption metric associated with the second measurement mode, or both, and wherein the second setting is further based at least in part on first biometric data collected for the user satisfying the second quality metric.

In some examples, the baseline setting is a user-specific baseline setting that is based at least in part on second biometric data collected for the user.

In some examples, the receiver 735 may be configured as or otherwise support a means for receiving an indication of the second measurement mode selected by the user for the wearable device.

In some examples, the baseline setting is determined while the wearable device is operating in a first operating mode, and the measurement mode component 730 may be configured as or otherwise support a means for determining to update the measurement parameter to the second setting based at least in part on the wearable device operating in the first operating mode and the second quality metric being for the first operating mode.

In some examples, the second quality metric is associated with higher quality biometric data than the first quality metric. In some examples, the second power consumption metric is associated with higher power consumption than the first power consumption metric.

In some examples, the second quality metric is associated with lower quality biometric data than the first quality metric. In some examples, the second power consumption metric is associated with lower power consumption than the first power consumption metric.

In some examples, the measurement mode component 730 may be configured as or otherwise support a means for configuring, as part of switching from the first measurement mode to the second measurement mode, one or more optical transmitters of the wearable device to output infrared signals instead of visible light signals.

In some examples, the charge monitoring component 740 may be configured as or otherwise support a means for determining a time at which the user is likely to charge the wearable device, wherein the second setting is based at least in part on a remaining battery life of the wearable device and the time at which the user is likely to charge the wearable device.

In some examples, the time at which the user is likely to charge the wearable device is determined based at least in part on a past charging pattern for charging the wearable device.

In some examples, the time at which the user is likely to charge the wearable device is determined based at least in part on an indication of the time from the user.

In some examples, the measurement mode component 730 may be configured as or otherwise support a means for determining a type of biometric data for which diagnostic-grade quality is selected, wherein the second quality metric is equal to the diagnostic-grade quality for the type of biometric data and the diagnostic-grade quality comprises a highest quality setting supported by the wearable device for that type of biometric data.

In some examples, the measurement mode component 730 may be configured as or otherwise support a means for modifying a rate of performing scanning operations for one or more signal channels of the wearable device based at least in part on updating the measurement parameter from the baseline setting to the second setting, wherein a scanning operation comprises an evaluation of a performance metric of the one or more signal channels.

In some examples, the wearable device is in wireless communication with a user device, and the user insight component 745 may be configured as or otherwise support a means for determining, based at least on an interaction of the user with an application of the user device, an interest level of the user in a type of biometric data, wherein the second setting is based at least in part on the interest level in the type of biometric data.

In some examples, the wearable device is in wireless communication with a user device, and the performance component 750 may be configured as or otherwise support a means for determining an impact of the wearable device switching to the second measurement mode on a performance metric of the wearable device. In some examples, the wearable device is in wireless communication with a user device, and the transmitter 755 may be configured as or otherwise support a means for transmitting an indication of the impact to the user device.

In some examples, the measurement parameter comprises an activation status of an optical transmitter of the wearable device, an activation status of an optical receiver of the wearable device, an amount of current supplied to the optical transmitter, a color of an optical signal output by the optical transmitter, an activation status of a combination of an optical transmitter and an optical receiver associated with a signal channel, a sample rate of a sensor, a scan rate of signal channel, or any combination thereof.

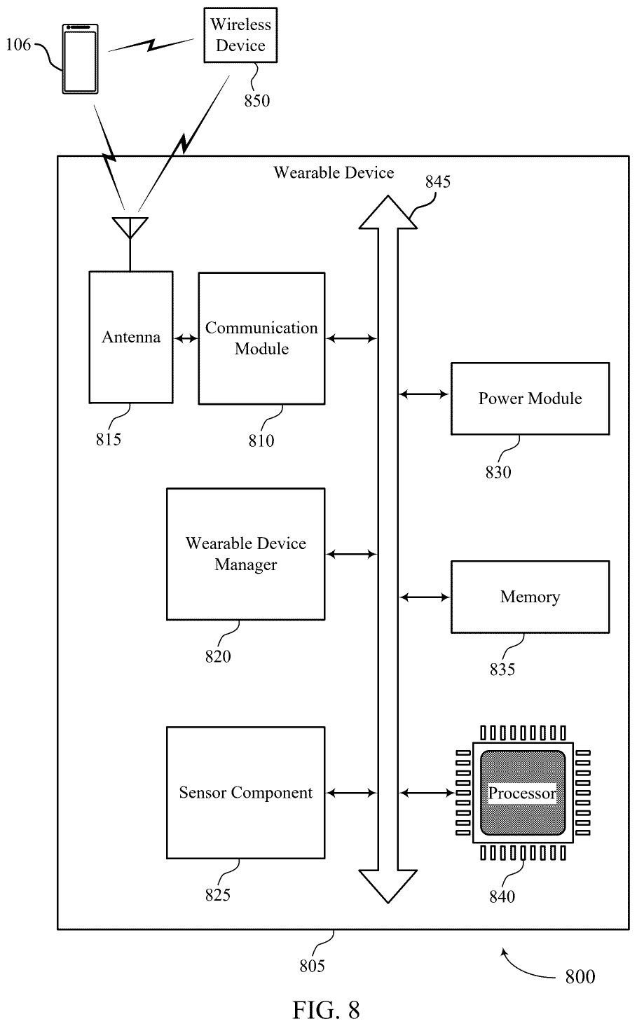

FIG. 8 shows a diagram of a system 800 including a device 805 that supports advanced measurement modes for a wearable device in accordance with aspects of the present disclosure. The device 805 may be an example of or include components of a device 605 as described herein. The device 805 may include an example of a wearable device 104, as described previously herein. The device 805 may include components for bi-directional communications including components for transmitting and receiving communications with a user device 106 and a server 110, such as a wearable device manager 820, a communication module 810, one or more antennas 815, a sensor component 825, a power module 830, at least one memory 835, at least one processor 840, and a wireless device 850. These components may be in electronic communication or otherwise coupled (e.g., operatively, communicatively, functionally, electronically, electrically) via one or more buses (e.g., a bus 845).

For example, the wearable device manager 820 may be configured as or otherwise support a means for determining, for a user while the wearable device is operating in a first measurement mode, a baseline setting for a measurement parameter for collecting biometric data from the user that satisfies a first quality metric and a first power consumption metric. The wearable device manager 820 may be configured as or otherwise support a means for updating, as part of switching from the first measurement mode to a second measurement mode, the measurement parameter from the baseline setting to a second setting that is based at least in part on a second quality metric associated with the second measurement mode, a second power consumption metric associated with the second measurement mode, or both, and wherein the second setting is further based at least in part on first biometric data collected for the user satisfying the second quality metric.

By including or configuring the wearable device manager 820 in accordance with examples as described herein, the device 805 may support techniques for improved user experience.

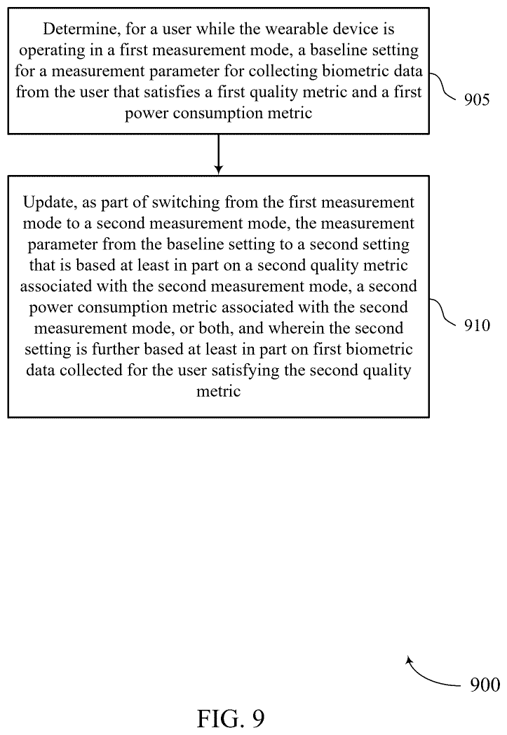

FIG. 9 shows a flowchart illustrating a method 900 that supports advanced measurement modes for a wearable device in accordance with aspects of the present disclosure. The operations of the method 900 may be implemented by a wearable device or its components as described herein. For example, the operations of the method 900 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 905, the method may include determining, for a user while the wearable device is operating in a first measurement mode, a baseline setting for a measurement parameter for collecting biometric data from the user that satisfies a first quality metric and a first power consumption metric. The operations of 905 may be performed in accordance with examples as disclosed herein. In some examples, aspects of the operations of 905 may be performed by a baseline component 725 as described with reference to FIG. 7.

At 910, the method may include updating, as part of switching from the first measurement mode to a second measurement mode, the measurement parameter from the baseline setting to a second setting that is based at least in part on a second quality metric associated with the second measurement mode, a second power consumption metric associated with the second measurement mode, or both, and wherein the second setting is further based at least in part on first biometric data collected for the user satisfying the second quality metric. The operations of 910 may be performed in accordance with examples as disclosed herein. In some examples, aspects of the operations of 910 may be performed by a measurement mode component 730 as described with reference to FIG. 7.

It should be noted that the methods described above describe possible implementations, and that the operations and the steps may be rearranged or otherwise modified and that other implementations are possible. Furthermore, aspects from two or more of the methods may be combined.

A method by an apparatus is described. The method may include determining, for a user while the wearable device is operating in a first measurement mode, a baseline setting for a measurement parameter for collecting biometric data from the user that satisfies a first quality metric and a first power consumption metric and updating, as part of switching from the first measurement mode to a second measurement mode, the measurement parameter from the baseline setting to a second setting that is based at least in part on a second quality metric associated with the second measurement mode, a second power consumption metric associated with the second measurement mode, or both, and wherein the second setting is further based at least in part on first biometric data collected for the user satisfying the second quality metric.

An apparatus is described. The apparatus may include one or more memories storing processor executable code, and one or more processors coupled with the one or more memories. The one or more processors may individually or collectively be operable to execute the code to cause the apparatus to determine, for a user while the wearable device is operating in a first measurement mode, a baseline setting for a measurement parameter for collecting biometric data from the user that satisfies a first quality metric and a first power consumption metric and update, as part of switching from the first measurement mode to a second measurement mode, the measurement parameter from the baseline setting to a second setting that is based at least in part on a second quality metric associated with the second measurement mode, a second power consumption metric associated with the second measurement mode, or both, and wherein the second setting is further based at least in part on first biometric data collected for the user satisfying the second quality metric.

Another apparatus is described. The apparatus may include means for determining, for a user while the wearable device is operating in a first measurement mode, a baseline setting for a measurement parameter for collecting biometric data from the user that satisfies a first quality metric and a first power consumption metric and means for updating, as part of switching from the first measurement mode to a second measurement mode, the measurement parameter from the baseline setting to a second setting that is based at least in part on a second quality metric associated with the second measurement mode, a second power consumption metric associated with the second measurement mode, or both, and wherein the second setting is further based at least in part on first biometric data collected for the user satisfying the second quality metric.

A non-transitory computer-readable medium storing code is described. The code may include instructions executable by one or more processors to determine, for a user while the wearable device is operating in a first measurement mode, a baseline setting for a measurement parameter for collecting biometric data from the user that satisfies a first quality metric and a first power consumption metric and update, as part of switching from the first measurement mode to a second measurement mode, the measurement parameter from the baseline setting to a second setting that is based at least in part on a second quality metric associated with the second measurement mode, a second power consumption metric associated with the second measurement mode, or both, and wherein the second setting is further based at least in part on first biometric data collected for the user satisfying the second quality metric.

In some examples of the method, apparatus, and non-transitory computer-readable medium described herein, the baseline setting may be a user-specific baseline setting that may be based at least in part on second biometric data collected for the user.

Some examples of the method, apparatus, and non-transitory computer-readable medium described herein may further include operations, features, means, or instructions for receiving an indication of the second measurement mode selected by the user for the wearable device.

In some examples of the method, apparatus, and non-transitory computer-readable medium described herein, the baseline setting may be determined while the wearable device may be operating in a first operating mode and the method, apparatuses, and non-transitory computer-readable medium may include further operations, features, means, or instructions for determining to update the measurement parameter to the second setting based at least in part on the wearable device operating in the first operating mode and the second quality metric being for the first operating mode.

In some examples of the method, apparatus, and non-transitory computer-readable medium described herein, the second quality metric may be associated with higher quality biometric data than the first quality metric and the second power consumption metric may be associated with higher power consumption than the first power consumption metric.

In some examples of the method, apparatus, and non-transitory computer-readable medium described herein, the second quality metric may be associated with lower quality biometric data than the first quality metric and the second power consumption metric may be associated with lower power consumption than the first power consumption metric.

In some examples of the method, apparatus, and non-transitory computer-readable medium described herein, configuring, as part of switching from the first measurement mode to the second measurement mode, one or more optical transmitters of the wearable device to output infrared signals instead of visible light signals.

Some examples of the method, apparatus, and non-transitory computer-readable medium described herein may further include operations, features, means, or instructions for determining a time at which the user may be likely to charge the wearable device, wherein the second setting may be based at least in part on a remaining battery life of the wearable device and the time at which the user may be likely to charge the wearable device.

In some examples of the method, apparatus, and non-transitory computer-readable medium described herein, the time at which the user may be likely to charge the wearable device may be determined based at least in part on a past charging pattern for charging the wearable device.

In some examples of the method, apparatus, and non-transitory computer-readable medium described herein, the time at which the user may be likely to charge the wearable device may be determined based at least in part on an indication of the time from the user.

Some examples of the method, apparatus, and non-transitory computer-readable medium described herein may further include operations, features, means, or instructions for determining a type of biometric data for which diagnostic-grade quality may be selected, wherein the second quality metric may be equal to the diagnostic-grade quality for the type of biometric data and the diagnostic-grade quality comprises a highest quality setting supported by the wearable device for that type of biometric data.

Some examples of the method, apparatus, and non-transitory computer-readable medium described herein may further include operations, features, means, or instructions for modifying a rate of performing scanning operations for one or more signal channels of the wearable device based at least in part on updating the measurement parameter from the baseline setting to the second setting, wherein a scanning operation comprises an evaluation of a performance metric of the one or more signal channels.

In some examples of the method, apparatus, and non-transitory computer-readable medium described herein, the wearable device may be in wireless communication with a user device and the method, apparatuses, and non-transitory computer-readable medium may include further operations, features, means, or instructions for determining, based at least on an interaction of the user with an application of the user device, an interest level of the user in a type of biometric data, wherein the second setting may be based at least in part on the interest level in the type of biometric data.

In some examples of the method, apparatus, and non-transitory computer-readable medium described herein, the wearable device may be in wireless communication with a user device and the method, apparatuses, and non-transitory computer-readable medium may include further operations, features, means, or instructions for determining an impact of the wearable device switching to the second measurement mode on a performance metric of the wearable device and transmitting an indication of the impact to the user device.

In some examples of the method, apparatus, and non-transitory computer-readable medium described herein, the measurement parameter comprises an activation status of an optical transmitter of the wearable device, an activation status of an optical receiver of the wearable device, an amount of current supplied to the optical transmitter, a color of an optical signal output by the optical transmitter, an activation status of a combination of an optical transmitter and an optical receiver associated with a signal channel, a sample rate of a sensor, a scan rate of signal channel, or any combination thereof.

Another apparatus is described. The apparatus may include a wearable device configured to, determine, for a user while the wearable device is operating in a first measurement mode and based at least in part on first biometric data collected for the user, a user-specific baseline setting for a measurement parameter for collecting biometric data from the user that satisfies a first quality metric and a first power consumption metric, update, as part of switching from the first measurement mode to a second measurement mode, the measurement parameter from the user-specific baseline setting to a second setting that is based at least in part on a second quality metric associated with the second measurement mode, a second power consumption metric associated with the second measurement mode, or both, and wherein the second setting is further based at least in part on second biometric data collected for the user satisfying second quality metric, a user device in electronic communication with the wearable device, the user device configured to, determine an impact of the wearable device switching to the second measurement mode on a performance metric of the wearable device, and display, by a graphical user interface, an indication of the impact of the wearable device switching to the second measurement mode on the performance metric of the wearable device.

In some examples of the apparatus, indication comprises an indication of a remaining battery life of the wearable device in the second measurement mode, an indication of the second quality metric, or both.

In some examples of the apparatus, the user device may be further configured to display, concurrently by the graphical user interface, an indication of the second measurement mode and a third measurement mode for the user to select between and transmit, to the wearable device, an indication of the second measurement mode based at least in part on the second measurement mode being selected by the user.

In some examples of the apparatus, the user device may be further configured to display, by the graphical user interface, an indication of an impact of the wearable device switching to the third measurement mode on the performance metric of the wearable device.

In some examples of the apparatus, the user device may be further configured to display, by the graphical user interface, a prompt suggesting that the user select the second measurement mode and transmit, to the wearable device, an indication of the second measurement mode based at least in part on the second measurement mode being selected by the user.

In some examples of the apparatus, the user device may be further configured to display, by the graphical user interface, a reminder that the wearable device may be using the second measurement mode.

In some examples of the apparatus, the user device may be further configured to display, by the graphical user interface, an indication that third biometric data collected by the wearable device was collected using the second measurement mode.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method of operating a wearable device, comprising:
determining, for a user while the wearable device is operating in a first measurement mode, a baseline setting for a measurement parameter for collecting biometric data from the user that satisfies a first quality metric and a first power consumption metric; and
updating, as part of switching from the first measurement mode to a second measurement mode, the measurement parameter from the baseline setting to a second setting that is based at least in part on a second quality metric associated with the second measurement mode, a second power consumption metric associated with the second measurement mode, or both, and wherein the second setting is further based at least in part on first biometric data collected for the user satisfying the second quality metric.

2. The method of claim 1, wherein the baseline setting is a user-specific baseline setting that is based at least in part on second biometric data collected for the user.

3. The method of claim 1, further comprising:
receiving an indication of the second measurement mode selected by the user for the wearable device.

4. The method of claim 1, wherein the baseline setting is determined while the wearable device is operating in a first operating mode, and wherein the first quality metric is for the first operating mode, the method further comprising:
determining to update the measurement parameter to the second setting based at least in part on the wearable device operating in the first operating mode and the second quality metric being for the first operating mode.

5. The method of claim 1, wherein the second quality metric is associated with higher quality biometric data than the first quality metric, and wherein the second power consumption metric is associated with higher power consumption than the first power consumption metric.

6. The method of claim 1, wherein the second quality metric is associated with lower quality biometric data than the first quality metric, and wherein the second power consumption metric is associated with lower power consumption than the first power consumption metric.

7. The method of claim 1, further comprising:
configuring, as part of switching from the first measurement mode to the second measurement mode, one or more optical transmitters of the wearable device to output infrared signals instead of visible light signals.

8. The method of claim 1, further comprising:
determining a time at which the user is likely to charge the wearable device, wherein the second setting is based at least in part on a remaining battery life of the wearable device and the time at which the user is likely to charge the wearable device.

9. The method of claim 8, wherein the time at which the user is likely to charge the wearable device is determined based at least in part on a past charging pattern for charging the wearable device.

10. The method of claim 8, wherein the time at which the user is likely to charge the wearable device is determined based at least in part on an indication of the time from the user.

11. The method of claim 1, further comprising:
determining a type of biometric data for which diagnostic-grade quality is selected, wherein the second quality metric is equal to the diagnostic-grade quality for the type of biometric data and the diagnostic-grade quality comprises a highest quality setting supported by the wearable device for that type of biometric data.

12. The method of claim 1, further comprising:
modifying a rate of performing scanning operations for one or more signal channels of the wearable device based at least in part on updating the measurement parameter from the baseline setting to the second setting, wherein a scanning operation comprises an evaluation of a performance metric of the one or more signal channels.

13. The method of claim 1, wherein the wearable device is in wireless communication with a user device, the method further comprising:
determining, based at least on an interaction of the user with an application of the user device, an interest level of the user in a type of biometric data, wherein the second setting is based at least in part on the interest level in the type of biometric data.

14. The method of claim 1, wherein the wearable device is in wireless communication with a user device, the method further comprising:
determining an impact of the wearable device switching to the second measurement mode on a performance metric of the wearable device; and
transmitting an indication of the impact to the user device.

15. The method of claim 1, wherein the measurement parameter comprises an activation status of an optical transmitter of the wearable device, an activation status of an optical receiver of the wearable device, an amount of current supplied to the optical transmitter, a color of an optical signal output by the optical transmitter, an activation status of a combination of an optical transmitter and an optical receiver associated with a signal channel, a sample rate of a sensor, a scan rate of signal channel, or any combination thereof.

16. A system, comprising:
a wearable device configured to:
determine, for a user while the wearable device is operating in a first measurement mode and based at least in part on first biometric data collected for the user, a user-specific baseline setting for a measurement parameter for collecting biometric data from the user that satisfies a first quality metric and a first power consumption metric; and
update, as part of switching from the first measurement mode to a second measurement mode, the measurement parameter from the user-specific baseline setting to a second setting that is based at least in part on a second quality metric associated with the second measurement mode, a second power consumption metric associated with the second measurement mode, or both, and wherein the second setting is further based at least in part on second biometric data collected for the user satisfying second quality metric; and
a user device in electronic communication with the wearable device, the user device configured to:
determine an impact of the wearable device switching to the second measurement mode on a performance metric of the wearable device; and
display, by a graphical user interface, an indication of the impact of the wearable device switching to the second measurement mode on the performance metric of the wearable device.

17. The system of claim 16, wherein indication comprises an indication of a remaining battery life of the wearable device in the second measurement mode, an indication of the second quality metric, or both.

18. The system of claim 16, wherein the user device is further configured to:
display, concurrently by the graphical user interface, an indication of the second measurement mode and a third measurement mode for the user to select between; and
transmit, to the wearable device, an indication of the second measurement mode based at least in part on the second measurement mode being selected by the user.

19. The system of claim 18, wherein the user device is further configured to:
display, by the graphical user interface, an indication of an impact of the wearable device switching to the third measurement mode on the performance metric of the wearable device.

20. The system of claim 16, wherein the user device is further configured to:
display, by the graphical user interface, a prompt suggesting that the user select the second measurement mode; and
transmit, to the wearable device, an indication of the second measurement mode based at least in part on the second measurement mode being selected by the user.

21. The system of claim 16, wherein the user device is further configured to:
display, by the graphical user interface, a reminder that the wearable device is using the second measurement mode.

22. The system of claim 16, wherein the user device is further configured to:
display, by the graphical user interface, an indication that third biometric data collected by the wearable device was collected using the second measurement mode.
